(19) United States
(12) Patent Application Publication
(10) Pub. No.: US 2025/0064305 A1
(43) Pub. Date: Feb. 27, 2025

(54) LENS POSITION CONTROL DEVICE AND ENDOSCOPE APPARATUS

(71) Applicant: FUJIFILM Corporation, Tokyo (JP)

(72) Inventors: Tomonori YOKOTA, Kanagawa (JP); Satoru TSUTO, Kanagawa (JP)

(73) Assignee: FUJIFILM Corporation, Tokyo (JP)

(21) Appl. No.: 18/764,098

(22) Filed: Jul. 3, 2024

(30) Foreign Application Priority Data
Aug. 25, 2023 (JP) 2023-137531

(51) Int. Cl. A61B 1/00; A61B 1/06

(52) U.S. Cl. CPC A61B 1/0019; A61B 1/00006; A61B 1/00013; A61B 1/0669

(57) ABSTRACT

A lens position control device is a lens position control device that controls a position of a variable magnification lens of a zooming optical system provided at an endoscope and includes a DC motor with a brush that generates a drive force for changing the position of the variable magnification lens, a drive circuit that drives the DC motor with a brush by repeating an application state where a drive voltage is applied between a pair of terminals of the DC motor with a brush and a release state where the terminals are released from each other, and a processor that controls the position of the variable magnification lens based on a time-integrated value of a counter-electromotive force generated at the DC motor with a brush in the release state.

### CROSS-REFERENCE TO RELATED APPLICATIONS

The present application claims priority under 35 U.S.C. § 119 to Japanese Patent Application No. 2023-137531 filed on Aug. 25, 2023. The above application is hereby expressly incorporated by reference, in its entirety, into the present application.

### BACKGROUND

### 1. Technical Field

The present disclosed technology relates to a lens position control device and an endoscope apparatus.

### 2. Description of the Related Art

An endoscope that includes an insertion part to be inserted into a body of a subject and an imaging unit disposed at a distal end portion of the insertion part to image an observation site in the body of the subject is known. The imaging unit includes an imaging element and an optical system that forms an image of the observation site on the imaging element. The optical system of the endoscope also includes a zooming optical system having a zoom function. In this case, a zoom mechanism that moves a variable magnification lens of the zooming optical system is provided at the endoscope.

The variable magnification lens is a movable lens that moves in an optical axis direction and changes an imaging magnification of the zooming optical system by moving in the optical axis direction. The zoom mechanism comprises a motor that generates a drive force for driving the variable magnification lens and a drive force transmission mechanism that transmits a drive force of the motor to the variable magnification lens through a wire or the like. In a case where focal lengths of the optical system are switched stepwise to a plurality of focal lengths set in advance as in so-called step zoom, it is necessary to move the variable magnification lens to target positions corresponding to the plurality of focal lengths. Therefore, a lens position control device that controls a position of the variable magnification lens through control of a motor is provided at the zoom mechanism (for example, see JP5219931B and JP6140362B).

The position of the variable magnification lens is controlled through rotation control of the motor, but there are various methods as a control method thereof. For example, there is a method of detecting the amount of rotation of the motor by detecting the rotation of the motor through a sensor such as an encoder and feeding back a detection signal. However, in the feedback control, a signal line for receiving the detection signal from the sensor is required and is not preferable in a case of an endoscope in which it is desired to reduce the number of wiring lines in a small-diameter tubular portion as much as possible. In addition, there also is a method of using a stepping motor that can accurately control the amount of rotation by counting the number of input driving pulses, but the stepping motor is disadvantageous in terms of cost and size compared to a direct current motor, in a case of being used in the endoscope.

Thus, the lens position control devices described in JP5219931B and JP6140362B use a sensorless direct current (DC) motor without using a sensor for feedback control and control the position of the variable magnification lens through the rotation control of the DC motor. The lens position control device described in JP5219931B uses a brushless DC motor driven by using three-phase driving pulses. Then, by detecting a generation timing of a counter-electromotive force generated at the brushless DC motor, one rotation detection signal indicating that the DC motor has been rotated once is acquired. The lens position control device described in JP5219931B detects the amount of rotation of the DC motor based on the one rotation detection signal and controls the position of the variable magnification lens. In addition, the lens position control device described in JP6140362B controls the amount of rotation of the DC motor by counting a drive time for driving the DC motor and controls the position of the variable magnification lens.

### SUMMARY

However, since a three-phase brushless DC motor is used, the lens position control device described in JP5219931B has a problem in which configurations of a motor and a drive circuit are complicated compared to a case of using a DC motor with a brush. In addition, in the lens position control device disclosed in JP6140362B, since the amount of rotation of the DC motor is controlled by counting the drive time for driving the DC motor, there is a concern that, in a case where fluctuations occur in the rotation speed of the DC motor due to load fluctuations, the accuracy of position control of the variable magnification lens decreases.

The present disclosed technology provides a lens position control device and an endoscope apparatus that can control a lens position with a simple configuration and high accuracy compared to the related art.

According to an aspect of the present disclosed technology, in order to achieve the object, there is provided a lens position control device that controls a position of a variable magnification lens of a zooming optical system provided at an endoscope, the lens position control device comprising a DC motor with a brush that generates a drive force for changing the position of the variable magnification lens, a drive circuit that drives the DC motor with a brush by repeating an application state where a drive voltage is applied between a pair of terminals of the DC motor with a brush and a release state where the terminals are released from each other, and a processor that controls the position of the variable magnification lens based on a time-integrated value of a counter-electromotive force generated at the DC motor with a brush in the release state.

It is preferable that the processor is configured to acquire an average voltage obtained by averaging the drive voltage and the counter-electromotive force and acquire the time-integrated value of the counter-electromotive force based on the average voltage and a known driving pulse corresponding to the drive voltage.

It is preferable that a low-pass filter that outputs an output signal of the average voltage obtained by smoothing an input signal corresponding to each of waveforms of the drive voltage generated between the terminals and the counter-electromotive force to the processor is further included.

It is preferable that the processor is configured to perform step zoom control of stepwise changing a focal length of the zooming optical system by stepwise changing the position of the variable magnification lens to a plurality of target positions set in advance.

It is preferable that a correspondence relationship between the plurality of target positions of the variable magnification lens and the time-integrated value of the counter-electromotive force required in order to move the variable magnification lens to each of the target positions is stored in advance in a memory, and the processor is configured to control the position of the variable magnification lens based on the correspondence relationship acquired from the memory.

It is preferable that a drive force transmission mechanism including a torque wire that transmits the drive force of the DC motor with a brush to the variable magnification lens and a reducer that decelerates rotation of the DC motor with a brush to transmit the rotation to the torque wire and that has an output shaft which is connected to the torque wire and which rotates the torque wire about an axis is further included.

It is preferable that the variable magnification lens moves from a wide end to a telephoto end in a range in which an amount of rotation of the output shaft is within one rotation.

It is preferable that in a case where a rotational position of the output shaft in a case where the variable magnification lens is at one of the wide end or the telephoto end is defined as a first end point position, and the rotational position of the output shaft in a case where the variable magnification lens is at the other is defined a second end point position, a touching portion that regulates rotation of the output shaft by touching the output shaft at each of the first end point position and the second end point position, in which the processor is configured to monitor the counter-electromotive force while the DC motor with a brush is driving and detect that the variable magnification lens has reached the wide end or the telephoto end in a case where the counter-electromotive force reaches a value of a rotation stop state of the DC motor with a brush.

It is preferable that the drive circuit drives the DC motor with a brush by repeating three states including a short-circuit state where the terminals are short-circuited, in addition to the application state and the release state.

It is preferable that the processor is configured to monitor the counter-electromotive force while the DC motor with a brush is driving and use the counter-electromotive force in control.

It is preferable that the processor is configured to control a rotation speed of the DC motor with a brush by adjusting an application time proportion of the drive voltage through the drive circuit based on a result of comparing the counter-electromotive force to a reference value set in advance.

It is preferable that the processor is configured to detect an abnormality including at least one of an abnormality of the DC motor with a brush or disconnection of a wiring line between the drive circuit and the DC motor with a brush based on the counter-electromotive force.

According to another aspect of the present disclosed technology, there is provided an endoscope apparatus comprising an endoscope that is provided with a zooming optical system and a lens position control device that controls a position of a variable magnification lens of the zooming optical system, in which the lens position control device includes a DC motor with a brush that generates a drive force for changing a position of the variable magnification lens, a drive circuit that drives the DC motor with a brush by repeating an application state where a drive voltage is applied between a pair of terminals of the DC motor with a brush and a release state where the terminals are released from each other, and a processor configured to control the position of the variable magnification lens based on a time-integrated value of a counter-electromotive force generated at the DC motor with a brush in the release state.

With the present disclosed technology, compared to the related art, a lens position can be controlled with a simple configuration and high accuracy.

### BRIEF DESCRIPTION OF THE DRAWINGS

FIG. 1 is a view showing an overall configuration of an endoscope system.

FIG. 2 is a view showing main portions of a zoom mechanism.

FIG. 3 is a view showing a configuration of a motor controller.

FIG. 4 is a time chart of a terminal-to-terminal voltage of a DC motor.

FIG. 5 is a graph showing a relationship between a rotation speed of the DC motor and a counter-electromotive force.

FIG. 6 is a view showing processing of a processor.

FIG. 7 is a view showing lens target position information.

FIG. 8 is a flowchart showing procedures of lens position control.

FIG. 9 is a view showing effects of a first embodiment.

FIG. 10 is a flowchart showing procedures of end point detection.

FIG. 11 is a time chart of a terminal-to-terminal voltage of a DC motor in a second embodiment.

FIG. 12 is a view showing a time proportion of a short-circuit section.

FIG. 13 is a view showing a configuration of a motor controller of a third embodiment.

FIG. 14 is a view showing an example of processing of detecting a counter-electromotive force.

FIG. 15 is a view showing another example of the processing of detecting the counter-electromotive force.

FIG. 16 is a flowchart showing procedures of speed control based on the counter-electromotive force.

FIG. 17 is a flowchart showing procedures of abnormality detection based on the counter-electromotive force.

### DETAILED DESCRIPTION OF THE PREFERRED EMBODIMENTS

### First Embodiment

### Overall Configuration of Endoscope System

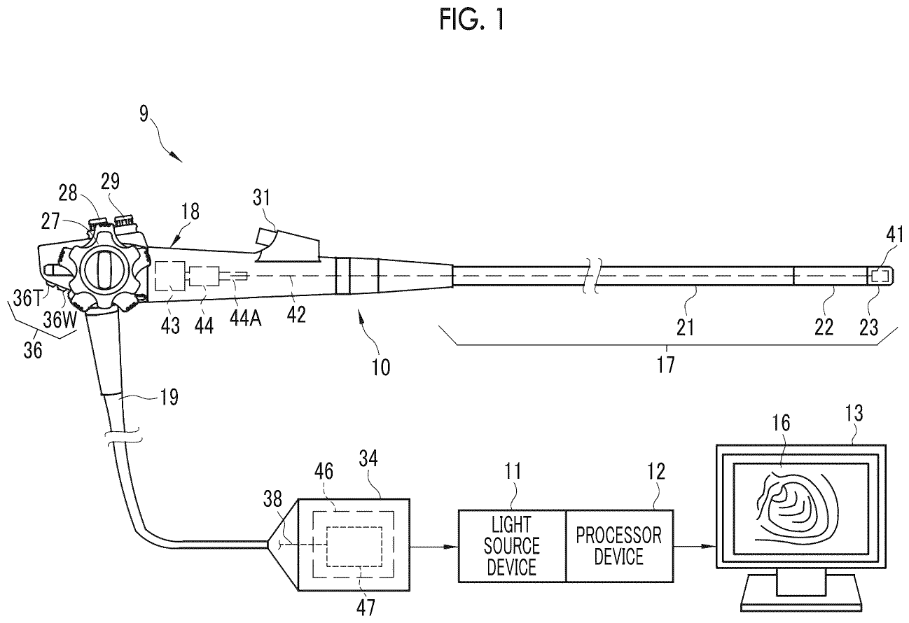

FIG. 1 is a schematic view showing an overall configuration of an endoscope system 9 to which a lens position control device and an endoscope apparatus according to an embodiment of the present disclosed technology are applied. As shown in FIG. 1, the endoscope system 9 comprises an endoscope 10, a light source device 11, a processor device 12, and a monitor 13. The endoscope system 9 is used in endoscopy in a body of a subject such as a patient. The endoscope 10 is, for example, an endoscope to be inserted into a digestive tract such as a large intestine and an esophagus and is a soft endoscope having flexibility. The endoscope 10 has an insertion part 17 that is inserted into the digestive tract, an operating part 18 that is consecutively installed on a proximal end side of the insertion part 17 and that is held by an operator for performing various types of operations, and a universal cord 19 that is consecutively installed on the operating part 18.

The light source device 11 supplies illumination light for illuminating an observation site in the body to the endoscope 10. The processor device 12 processes an image captured by the endoscope 10 to generate an observation image 16. The observation image 16 is displayed on the monitor 13. A doctor who is a user proceeds with endoscopy while checking the observation image 16. The observation image 16 displayed on the monitor 13 is basically a motion picture, but a still image can also be displayed as the observation image 16 as necessary.

The insertion part 17 is a small-diameter and long tubular portion and is composed of a soft portion 21, a bendable part 22, and a distal end portion 23 which are joined to each other in order from the proximal end side toward a distal end side. The soft portion 21 has flexibility. The bendable part 22 is a part that can be bent by operating the operating part 18. An imaging unit 41 and the like are disposed at the distal end portion 23. In addition, although not shown, on a distal end surface of the distal end portion 23, an illumination window through which the observation site is illuminated with illumination light, an observation window to which subject light obtained by a subject reflecting the illumination light is incident, a treatment tool outlet through which a treatment tool protrudes, and a washing nozzle for washing the observation window by jetting a gas and water to the observation window are provided.

A light guide (not shown), a signal cable, an operation wire (not shown), a treatment tool insertion pipe line (not shown), and the like are provided at the insertion part 17. The light guide is projected from the universal cord 19 and guides illumination light supplied from the light source device 11 to the illumination window of the distal end portion 23. The signal cable is used in supplying power to the imaging unit 41 in addition to communication of an image signal from the imaging unit 41 and a control signal for controlling the imaging unit 41. The signal cable is also projected from the universal cord 19 and is arranged up to the distal end portion 23, like the light guide.

The operation wire is a wire for operating the bendable part 22 and is arranged from the operating part 18 to the bendable part 22. The treatment tool insertion pipe line is a pipe line for inserting a treatment tool (not shown), such as a forceps, and is arranged from the operating part 18 to the distal end portion 23. In addition, a fluid tube for supplying air and water is provided at the insertion part 17. Air and water for washing the observation window are supplied to the distal end portion 23 through the fluid tube.

In addition, a torque wire 42 is arranged in the insertion part 17. The imaging unit 41 has a zoom function that can change an imaging magnification. As will be described later, the torque wire 42 is used in order to change the imaging magnification of the imaging unit 41. The torque wire 42 has flexibility and can be rotated around an axis thereof. The torque wire 42 is projected from the distal end portion 23 where the imaging unit 41 is provided to the operating part 18 on the proximal end side.

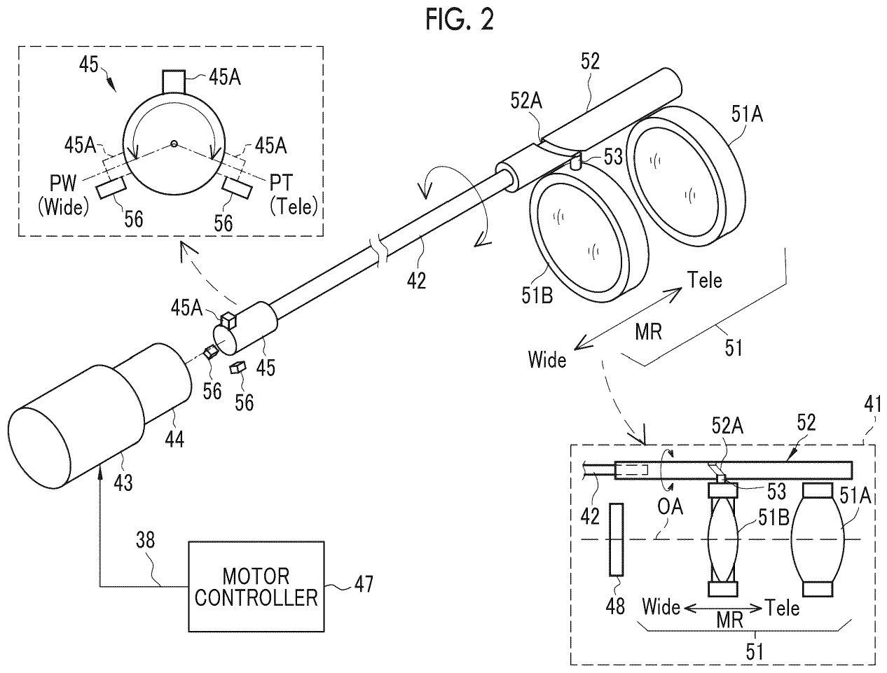

A DC motor 43 that rotates the torque wire 42 and a reducer 44 that decelerates the rotation of the DC motor 43 are provided at the operating part 18. One end of the torque wire 42 on the proximal end side is connected to an output shaft 45 of the reducer 44. The DC motor 43 is a DC motor with a brush. The DC motor 43 generates a drive force for changing the position of a variable magnification lens 51B (see FIG. 2) in the imaging unit 41 as will be described later. As is well known, the DC motor with a brush is a motor that has a brush that comes into contact with a commutator connected to a coil wound around a rotor. The DC motor with a brush has a simple configuration and a simple drive circuit configuration compared to a brushless DC motor. The DC motor 43 is an example of a “DC motor with a brush” according to the embodiment of the present disclosed technology.

In addition, various types of operation members operated by the operator are provided at the operating part 18. Specifically, two types of bending operation knobs 27, an air/water supply button 28, a suction button 29, and a zoom operation button 36 are provided at the operating part 18. Each of the two types of bending operation knobs 27 is connected to the operation wire and is used in a right-left bending operation and an up-down bending operation of the bendable part 22. The zoom operation button 36 is operated in a case of changing the imaging magnification of the imaging unit 41. The zoom operation button 36 includes a wide side button 36W for changing the imaging magnification to a wide side and a telephoto side button 36T for changing the imaging magnification to a telephoto side. In addition, a treatment tool inlet port 31 that is an inlet for the treatment tool insertion pipe line is provided at the operating part 18.

The universal cord 19 is a connection cord for connecting the endoscope 10 to the light source device 11. The universal cord 19 includes a signal cable (not shown), a light guide (not shown), and a fluid tube (not shown). In addition, a connector 34 connected to the light source device 11 is provided at an end part of the universal cord 19.

For example, a control substrate 46 for controlling the endoscope 10 is provided at the connector 34. In a case where the connector 34 is connected to the light source device 11, the control substrate 46 is electrically connected to the light source device 11 and the processor device 12. Accordingly, power, a control signal, illumination light, a gas, and water necessary for the operation of the endoscope 10 are supplied from the light source device 11 to the endoscope 10. In addition, an image signal of the observation site acquired by the imaging unit 41 of the distal end portion 23 is transmitted from the endoscope 10 to the light source device 11.

In addition, a motor controller 47 that controls the DC motor 43 is provided on the control substrate 46. A signal line 38 that connects the motor controller 47 and the DC motor 43 to each other is arranged in the universal cord 19. A drive signal is transmitted from the motor controller 47 to the DC motor 43 through the signal line 38.

As shown in FIG. 2, the imaging unit 41 includes an imaging element 48 and a zooming optical system 51 having a zoom function as an imaging optical system. The imaging element 48 and the zooming optical system 51 are disposed along an optical axis OA. The imaging element 48 is an image sensor having an imaging surface on which a plurality of pixels composed of photoelectric conversion elements are two-dimensionally arranged and outputs an electric signal representing a subject image formed on the imaging surface.

The zooming optical system 51 includes, for example, an objective lens 51A and the variable magnification lens 51B. Luminous flux representing a subject image, which is an observation target, is incident to the objective lens 51A. The objective lens 51A collects the incident luminous flux and forms the subject image on the imaging surface of the imaging element 48. The variable magnification lens 51B is a movable lens for changing the imaging magnification of the zooming optical system 51. In a case where the variable magnification lens 51B moves along the optical axis OA, a focal length of the zooming optical system 51 changes, and the imaging magnification changes. The variable magnification lens 51B is provided to be movable between a wide end having the shortest focal length and a maximum angle of view and a telephoto end having the longest focal length and a minimum angle of view. At the wide end, the angle of view is wide but the imaging magnification is minimum, and at the telephoto end, the angle of view is narrow but the imaging magnification is maximum. An example in which the zooming optical system 51 is composed of two lenses is shown, but this is merely a schematic view, and the zooming optical system 51 is actually composed of two or more lenses in many cases, and the variable magnification lens 51B may also be composed of a plurality of lenses in some cases.

The variable magnification lens 51B changes a lens position in an optical axis OA direction as the torque wire 42 and a cam shaft 52 connected to one end of the torque wire 42 on the distal end side operate. The cam shaft 52 is a shaft extending along the optical axis OA, and a cam groove 52A is formed in a peripheral surface thereof. A cam pin 53 is provided in a lens holding frame of the variable magnification lens 51B, and the cam groove 52A and the cam pin 53 engage with each other. In a case where the cam shaft 52 is rotated about an axis by the rotation of the torque wire 42, an engagement position between the cam pin 53 and the cam groove 52A is displaced along the optical axis OA direction, and the variable magnification lens 51B moves along the optical axis OA. For example, in a case where the torque wire 42 rotates in a positive direction, the variable magnification lens 51B moves toward the telephoto end. In a case where the torque wire 42 rotates in a negative direction which is opposite to the positive direction, the variable magnification lens 51B moves toward the wide end.

A protruding portion 45A that protrudes radially is provided on a part of an outer peripheral surface of the output shaft 45 of the reducer 44. A touching portion 56 that touches the protruding portion 45A is provided around the output shaft 45. The touching portion 56 regulates a rotation range of the output shaft 45 by touching the protruding portion 45A. At two places, the touching portions 56 are provided. The rotation range of the output shaft 45 is set to be within one rotation (that is, 360°) by the touching portion 56. This is because, by setting the amount of rotation of the torque wire 42 about the axis to be within one rotation, the twisting of the torque wire 42 is suppressed. For example, the rotation range of the output shaft 45 is in a range of 300° to 350°. The variable magnification lens 51B moves from the wide end to the telephoto end in a range where the amount of rotation of the output shaft 45 is within one rotation. Such a rotation range of the torque wire 42 and such a movement range MR of the variable magnification lens 51B are defined by the cam groove 52A.

For example, a rotational position of the output shaft 45 in a case where the variable magnification lens 51B is at the wide end is a wide side end point position PW, and a rotational position of the output shaft 45 in a case where the variable magnification lens 51B is at the telephoto end is a telephoto side end point position PT. In this case, one of the two touching portions 56 touches the protruding portion 45A of the output shaft 45 at the wide side end point position PW to regulate the rotation of the output shaft 45. The other touching portion 56 touches the protruding portion 45A of the output shaft 45 at the telephoto side end point position PT to regulate the rotation of the output shaft 45. The wide side end point position PW and the telephoto side end point position PT are examples of a “first end point position” and a “second end point position” according to the embodiment of the present disclosed technology, respectively.

As described above, the torque wire 42 transmits the drive force of the DC motor 43 to the variable magnification lens 51B. The reducer 44 is a reducer that decelerates the rotation of the DC motor 43 and that transmits the rotation to the torque wire and has the output shaft 45 that is connected to the torque wire 42 and that rotates the torque wire 42 about the axis. The torque wire 42 and the reducer 44 are examples of a “drive force transmission mechanism” according to the embodiment of the present disclosed technology.

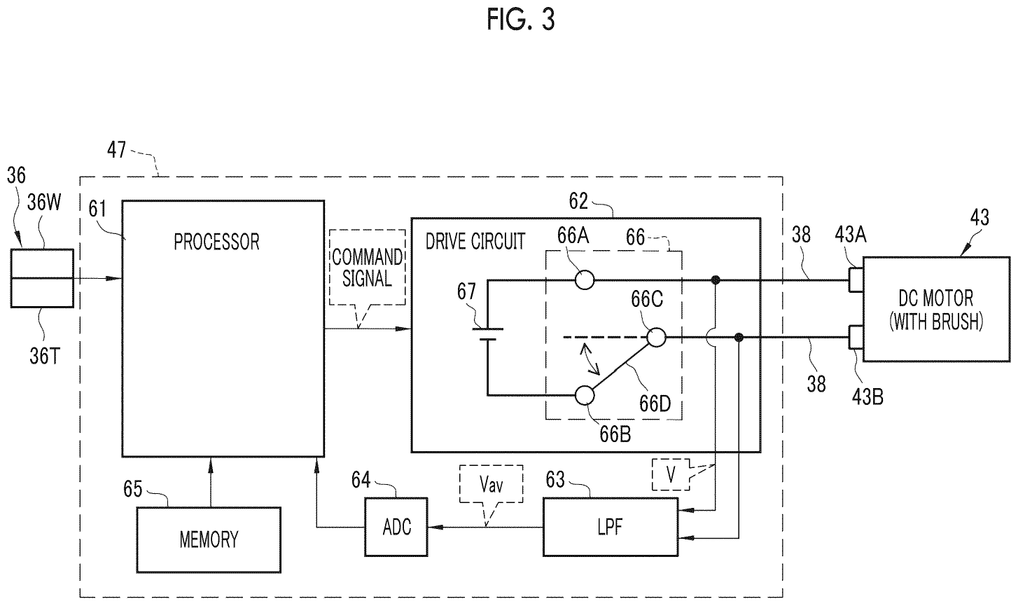

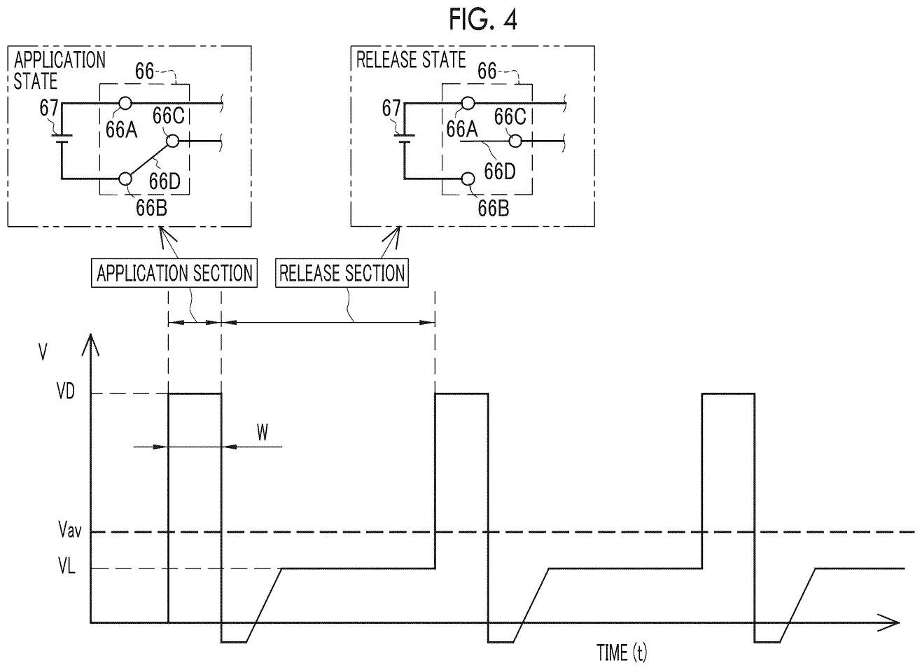

As shown in FIG. 3, the motor controller 47 includes a processor 61, a drive circuit 62, a low-pass filter (LPF) 63, an analog-to-digital converter (ADC) 64, and a memory 65. The drive circuit 62 drives the DC motor 43 by outputting a driving pulse as a drive signal. The drive circuit 62 generates a driving pulse by repeating an application state where a drive voltage VD (see FIG. 4) is applied between a pair of terminals 43A and 43B of the DC motor 43 and a release state where the terminals 43A and 43B are released from each other. Then, the drive circuit 62 drives the DC motor 43 in a pulse width modulation (PWM) mode in which the driving pulse is periodically input.

The drive circuit 62 includes a switch circuit 66 that generates a driving pulse. As schematically shown in FIG. 3, the switch circuit 66 has three contacts including a first contact 66A, a second contact 66B, and a third contact 66C. The first contact 66A is connected to a positive side of a power source 67, and the second contact 66B is connected to a negative side. The first contact 66A is connected to the terminal 43A of the DC motor 43, and the third contact 66C is connected to the other terminal 43B. One end of a switch 66D is connected to the third contact 66C.

As also shown in FIG. 4, a state where the other end of the switch 66D is connected to the second contact 66B is the application state. In the application state, the drive voltage VD of the power source 67 is applied between the terminals 43A and 43B of the DC motor 43. On the other hand, a state where the other end of the switch 66D is not connected to any of the first contact 66A or the second contact 66B is the release state. In the release state, the drive voltage VD of the power source 67 is not applied to the DC motor 43.

In the normal PWM mode, a short-circuit state where the other end of the switch 66D is connected to the first contact 66A is used instead of the release state, and a driving pulse is generated by repeating the application state and the short-circuit state. The PWM mode according to the embodiment of the present disclosed technology is different from the PWM mode of the related art in this point.

As shown in a time chart of a terminal-to-terminal voltage V generated between the terminals 43A and 43B of the DC motor 43 in FIG. 4, the application state and the release state are periodically repeated, so that the driving pulse is continuously generated. The driving pulse has a rectangular wave of which a wave height is the drive voltage VD. In the PWM mode, a duty ratio is changed by adjusting a pulse width W of the driving pulse in one period. Supply power from the power source 67 to the DC motor 43 can be changed. It is evident that the supply power increases as the pulse width W becomes wider, that is, an application section in which the application state continues becomes longer.

As shown in the time chart of FIG. 4, in the release state, the drive voltage VD is not applied to the DC motor 43, but a counter-electromotive force VL is generated in the DC motor 43 by the self-induction action of a coil of the DC motor 43. As is well known, the counter-electromotive force VL is a voltage generated in a direction of preventing a current change occurring in the coil of the DC motor 43. In a case where the application section is finished, the application of the drive voltage VD is finished, so that a current flowing into the coil of the DC motor 43 tends to decrease, but the counter-electromotive force VL is generated in a direction of preventing the current change. For this reason, the counter-electromotive force VL is lower than the drive voltage VD, but a voltage in a direction of continuing the rotation of the DC motor 43 is generated.

In the time chart of FIG. 4, the terminal-to-terminal voltage V drops immediately after the waveform of the driving pulse falls, which is an overshoot. In addition, the positive and negative of the drive voltage VD are reversed depending on a rotation direction of the DC motor 43.

In the time chart of FIG. 4, an average voltage Vav is an average value of the drive voltage VD and the counter-electromotive force VL in consideration of a time proportion in one period of the driving pulse. The average voltage Vav is defined as follows.

$$Vav \times ( t 1 + t 2 ) = VD \times t 1 + VL \times t 2 \qquad Equation ( 1 )$$

Through equation (1), the following is acquired.

$$Vav = ( VD \times t 1 + VL \times t 2 ) / ( t 1 + t 2 ) \qquad Equation ( 2 )$$

Herein, t1 and t2 are defined as follows. In a case where a total time of t1 and t2 is defined as a drive period of the DC motor 43, t1 is a total time of the application section in which the drive voltage VD is generated in the drive period, and t2 is a total time of the release section in which the counter-electromotive force VL is generated in the drive period. The left side of equation (1) represents power supplied to the DC motor 43 in a certain drive period (t1+t2), and driving the DC motor 43 in the PWM mode with the driving pulse of the drive voltage VD is equivalent to continuously inputting the average voltage Vav to the DC motor 43 and driving the DC motor 43.

In practice, since the counter-electromotive force VL fluctuates within the drive period, the counter-electromotive force VL in equations (1) and (2) is an average value in the drive period.

Returning to FIG. 3, the LPF 63 smoothes an input signal corresponding to the waveform of each of the terminal-to-terminal voltage V of the DC motor 43, that is, the drive voltage VD generated between the terminal 43A and the terminal 43B of the DC motor 43 and the counter-electromotive force VL and outputs an output signal, which is the smoothed average voltage Vav, to the processor 61 via the ADC 64. The LPF 63 is a known LPF composed of, for example, an RC filter in which a resistor and a capacitor are combined, an LC filter in which an inductor and a capacitor are combined, or the like.

The ADC 64 converts an analog signal, which is the average voltage Vav output by the LPF 63, into a digital signal and outputs the average voltage Vav, which is the converted digital signal, to the processor 61.

The processor 61 is, for example, a microcomputer, and an operation signal from the zoom operation button 36 is input to the processor 61. In a case where the operation signal is input, the processor 61 outputs a command signal for commanding rotation start or rotation stop of the DC motor 43 to the drive circuit 62. The drive circuit 62 starts or stops driving the DC motor 43 based on the command signal from the processor 61.

The processor 61 controls the position of the variable magnification lens 51B of the zooming optical system 51 based on an input operation signal. More specifically, the processor 61 performs step zoom control of stepwise changing the focal length of the zooming optical system 51 by stepwise changing the position of the variable magnification lens 51B to a plurality of target positions set in advance. In a case of performing the step zoom control, the variable magnification lens 51B needs to be moved to the target position and be stopped at the target position. Thus, it is necessary to control the amount of movement of the variable magnification lens 51B through control of the amount of rotation of the DC motor 43. The processor 61 controls the position of the variable magnification lens 51B based on a time-integrated value Q (VL) of the counter-electromotive force VL.

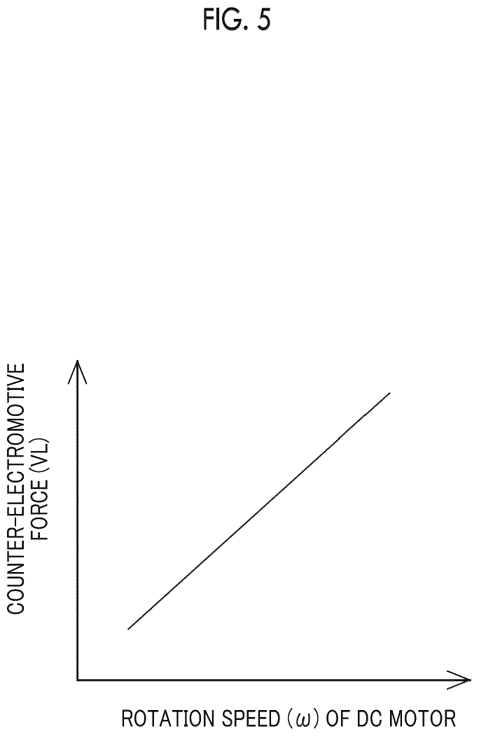

As shown in FIG. 5, the counter-electromotive force VL and a rotation speed ω of the DC motor 43 are correlated with each other. For example, the counter-electromotive force VL is proportional to the rotation speed ω of the DC motor 43. For this reason, the processor 61 can learn the amount of rotation of the DC motor 43 by detecting the time-integrated value Q (VL) of the counter-electromotive force VL during driving, and as a result, the amount of movement of the variable magnification lens 51B can be controlled.

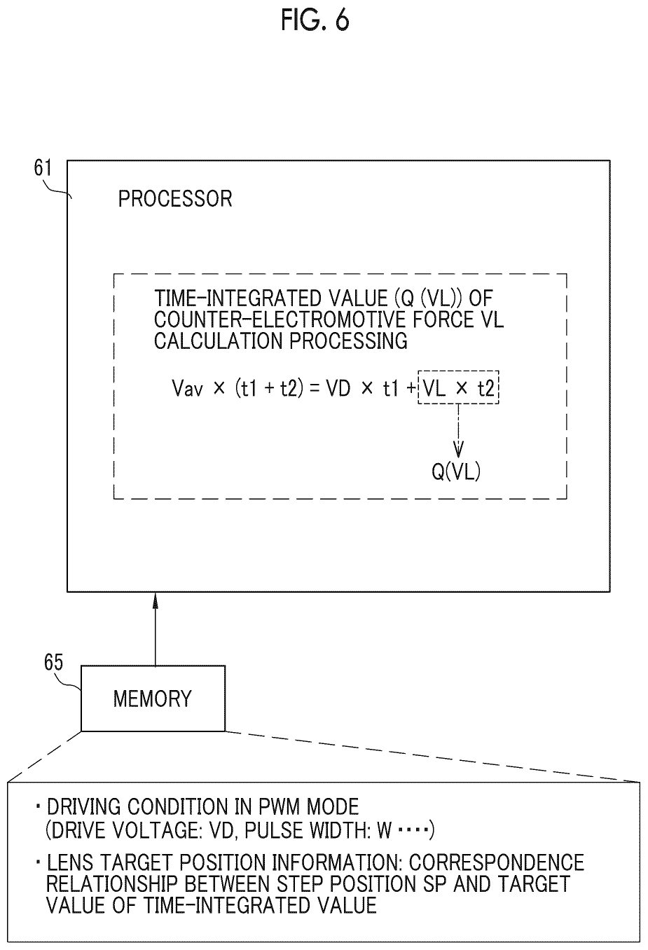

As shown in FIG. 6, in the present example, the processor 61 acquires the average voltage Vav obtained by averaging the drive voltage VD and the counter-electromotive force VL from the LPF 63 and calculates the time-integrated value Q (VL) of the counter-electromotive force VL based on the acquired average voltage Vav and a known driving pulse corresponding to the drive voltage VD. The memory 65 stores a driving condition in the PWM mode. The driving condition in the PWM mode includes the drive voltage VD and the pulse width W of a driving pulse. The processor 61 calculates the time-integrated value Q (VL) of the counter-electromotive force VL based on equation (1) by using the pieces of information and the average voltage Vav. The time-integrated value Q (VL) of the counter-electromotive force VL is an integrated value of times when the counter-electromotive force VL is generated in a driving period of the DC motor 43.

As shown in FIG. 5, since the counter-electromotive force VL represents the rotation speed of the DC motor 43, the time-integrated value Q (VL) of the counter-electromotive force VL represents the amount of rotation of the DC motor 43, and as a result, represents the amount of movement of the variable magnification lens 51B.

Further, the memory 65 stores lens target position information. The lens target position information is a correspondence relationship between a plurality of step positions SP, which are target positions to which the variable magnification lens 51B is to be moved in the step zoom, and a target value TQ (VL) of the time-integrated value for movement to each step position SP.

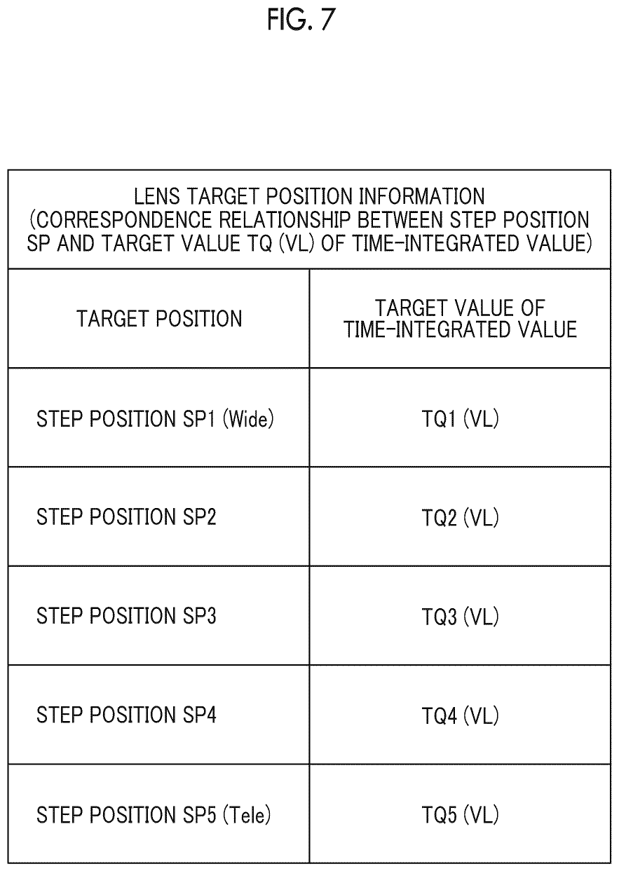

FIG. 7 is an example of lens target position information. In the example in FIG. 7, target values TQ1 (VL) to TQ5 (VL) of the time-integrated value are associated with the step positions SP1 to SP5, respectively. For example, the step position SP1 is a position corresponding to the wide end for the variable magnification lens 51B, and the step position SP5 is a position corresponding to the telephoto end for the variable magnification lens 51B.

Each of the target values TQ1 (VL) to TQ5 (VL) is the amount of movement from a reference position set in advance. For example, in a case where the reference position is the step position SP1, the target value TQ1 is “0”. In addition, the target value TQ2 corresponds to the amount of movement of the variable magnification lens 51B from the step position SP1, which is at the wide end, to the step position SP2. The target value TQ3 corresponds to the amount of movement of the variable magnification lens 51B from the step position SP1 to the step position SP3. The same applies to the target values TQ4 and TQ5.

Such lens target position information is created, for example, as follows. The DC motor 43 is driven to rotate the output shaft 45 from the wide side end point position PW to the telephoto side end point position PT. In response to the rotation, the variable magnification lens 51B also moves from the wide end to the telephoto end. While the DC motor 43 is driving, the processor 61 measures the time-integrated value Q (VL) of the counter-electromotive force VL and acquires a total of the time-integrated values Q (VL) from the wide side end point position PW to the telephoto side end point position PT. On the other hand, in a case where the variable magnification lens 51B is moved from the wide end to the telephoto end, a correlation between the amount of movement of the variable magnification lens 51B and the amount of change in the focal length is known as design information of the zooming optical system 51. That is, it is known how much movement changes the focal length. In addition, a focal length corresponding to each of the step positions SP1 to SP5 is determined. In a case where the total of the time-integrated values Q (VL) is known, the time-integrated value Q (VL) in a case where the variable magnification lens 51B is moved to each of the step positions SP1 to SP5 from the reference position can be acquired. Each of the time-integrated values Q (VL) of the step positions SP1 to SP5 is recorded as each target value TQ (VL).

In addition, the target value TQ (VL) of the time-integrated value corresponding to each of the step positions SP1 to SP5 can be acquired through the following method in addition to a method of acquiring lens target position information based on a total of the time-integrated values Q (VL). That is, the focal length is changed by moving the variable magnification lens 51B, but a focal length at the current position of the variable magnification lens 51B can be calculated from the size of a subject image formed on the imaging element 48 based on an image formation formula in a case where a subject distance is known. Since the focal length at each of the step positions SP1 to SP5 is determined, in a case where the focal length is calculated backward based on the position of the variable magnification lens 51B, the position of the variable magnification lens 51B can be detected from the size of the subject image. Through this method, the variable magnification lens 51B is moved to each of the step positions SP1 to SP5, and the time-integrated value Q (VL) required in order to move the variable magnification lens 51B to each position is detected. Then, each of the detected time-integrated values Q (VL) of the step positions SP1 to SP5 is recorded as each target value TQ (VL).

As described above, lens target position information is a correspondence relationship between the step positions SP1 to SP5, which are the plurality of target positions of the variable magnification lens 51B, and the time-integrated value Q (VL) of the counter-electromotive force VL required in order to move the variable magnification lens 51B to each of the target positions SP1 to SP5, that is, the target value TQ (VL). The processor 61 controls the position of the variable magnification lens 51B based on the lens target position information acquired from the memory 65.

In addition, since the DC motor 43 rotates in both the positive direction and the negative direction, the time-integrated value Q (VL) of the counter-electromotive force VL is a positive value in a case where the DC motor 43 rotates in the positive direction, and is a negative value in a case where the DC motor 43 rotates in the negative direction. The processor 61 learns the current position of the variable magnification lens 51B using the time-integrated value QL (VL) of the counter-electromotive force VL. For example, the processor 61 adds the positive time-integrated value Q (VL) of the counter-electromotive force VL in a case where the variable magnification lens 51B moves from the wide end toward the telephoto end with reference to an initial position of the variable magnification lens 51B. Then, in a case where the variable magnification lens 51B moves from the telephoto end toward the wide end, the negative time-integrated value Q (VL) of the counter-electromotive force VL is added. In such a manner, in a case where the initial position of the variable magnification lens 51B is set, the processor 61 can learn the current position of the variable magnification lens 51B by using the time-integrated value Q (VL) of the counter-electromotive force VL.

A configuration where the motor controller 47, the DC motor 43, the reducer 44, the torque wire 42, and the cam shaft 52 are combined is an example of a “lens position control device” according to the embodiment of the present disclosed technology. In addition, the endoscope 10 to which the “lens position control device” is applied is an example of the “endoscope apparatus” according to the embodiment of the present disclosed technology.

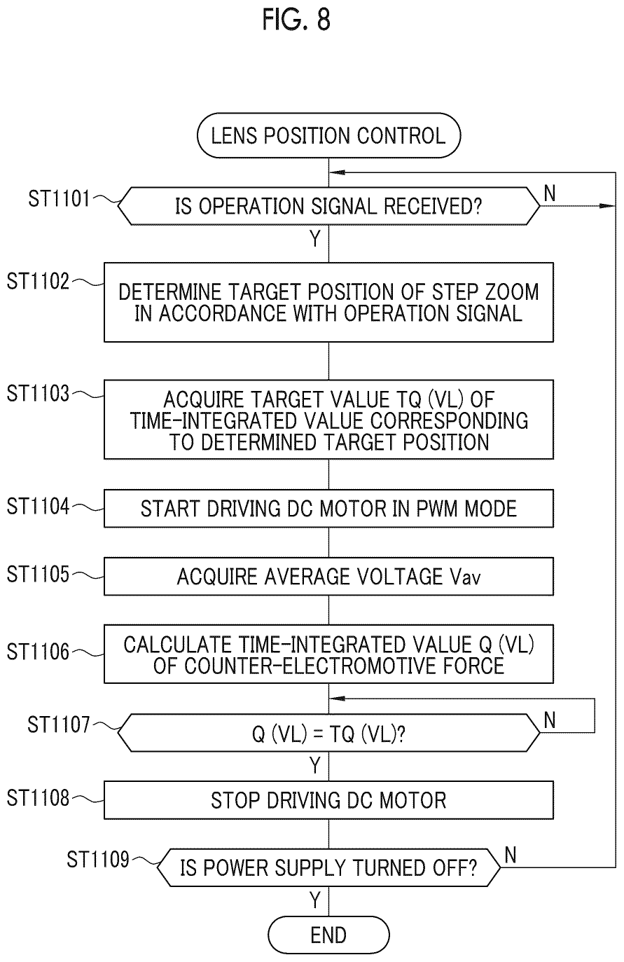

The actions of the configuration will be described with reference to a flowchart of lens position control shown in FIG. 8. In a case where the endoscope system 9 is started, the processor 61 starts lens position control processing. In step ST1101, the processor 61 waits for the input of an operation signal through the operation of the zoom operation button 36. In a case where the operation signal is input (Y in step ST1101), the processing proceeds to step ST1102.

In step ST1102, the processor 61 determines the target position of the step zoom in accordance with the operation signal. For example, in a case where the zoom operation button 36 is pressed once, the processor 61 determines the step position SP, which is the step position SP is moved by one stage, as a target position of the step zoom. More specifically, for example, in a case where the current position of the variable magnification lens 51B is the step position SP1 and the telephoto side button 36T is pressed once, the processor 61 determines the step position SP2, which is a position moved to the telephoto side by one stage from the step position SP1, as a target position.

Then, in step ST1103, the processor 61 acquires the target value TQ (VL) of the time-integrated value corresponding to the determined target position with reference to the lens target position information shown in FIG. 7. In a case where the step position SP1 is moved to the step position SP2, the target value TQ (VL) of the time-integrated value corresponding to the step position SP2 is acquired.

In step ST1104, the processor 61 starts driving the DC motor 43 in the PWM mode. In step ST1105, the processor 61 acquires the average voltage Vav while the DC motor 43 is driving.

In step ST1106, the processor 61 calculates the time-integrated value Q (VL) of the counter-electromotive force VL in the procedures shown in FIG. 8 based on the average voltage Vav.

In step ST1107, the processor 61 compares the time-integrated value Q (VL) to the target value TQ (VL) to determine whether or not both values match each other. In a case where it is determined that both values match each other (Y in step ST1107), the processor 61 stops driving the DC motor 43 in step ST1108. Accordingly, the variable magnification lens 51B can be moved to the step position SP corresponding to the input operation signal.

As described above, the “lens position control device” according to the embodiment of the present disclosed technology applied to the endoscope system 9 comprises the DC motor 43 with a brush that generates a drive force for changing the position of the variable magnification lens 51B of the zooming optical system 51, the drive circuit 62 that drives the DC motor 43 by repeating the application state where a drive voltage is applied between the pair of terminals of the DC motor 43 and the release state where the terminals are released from each other, and the processor 61 that controls the position of the variable magnification lens 51B based on the time-integrated value Q (VL) of the counter-electromotive force VL generated at the DC motor 43 in the release state. For this reason, compared to the related art, the lens position can be controlled with a simple configuration and high accuracy.

That is, first, since the DC motor 43, which is an example of the DC motor with a brush, is used, configurations of the DC motor 43 and the drive circuit 62 are simple compared to a case where the three-phase brushless DC motor described in JP5219931B is used. In addition, since the position of the variable magnification lens 51B is controlled based on the time-integrated value Q (VL) of the counter-electromotive force VL, the accuracy of the control of the lens position is also high compared to the technique disclosed in JP6140362B in which the amount of rotation of the DC motor is controlled by counting a drive time.

This is because, in a case where a load fluctuates due to various factors even with the same driving pulse applied to the DC motor 43, the rotation speed ω of the DC motor 43 fluctuates. The various factors include a change in a resistance value of the DC motor 43 caused by the environment temperature, temporal deterioration caused by the progress of abrasion of the DC motor 43, the reducer 44, and the like, and a change in the viscosity of grease of the reducer 44 caused by a change in the environment temperature or the elapse of time. However, since the counter-electromotive force VL has a correlation with the rotation speed ω of the DC motor 43 (for example, a proportional relationship as shown in FIG. 5), the time-integrated value Q (VL) of the counter-electromotive force VL for controlling the amount of rotation of the DC motor 43 and the amount of movement of the variable magnification lens 51B corresponding to the amount of rotation is a value in which fluctuations in the rotation speed ω caused by the load fluctuations are reflected. For this reason, with the lens position control device according to the embodiment of the present disclosed technology, the position of the variable magnification lens 51B can be accurately controlled even in a case where there are fluctuations in the rotation speed ω caused by the load fluctuations.

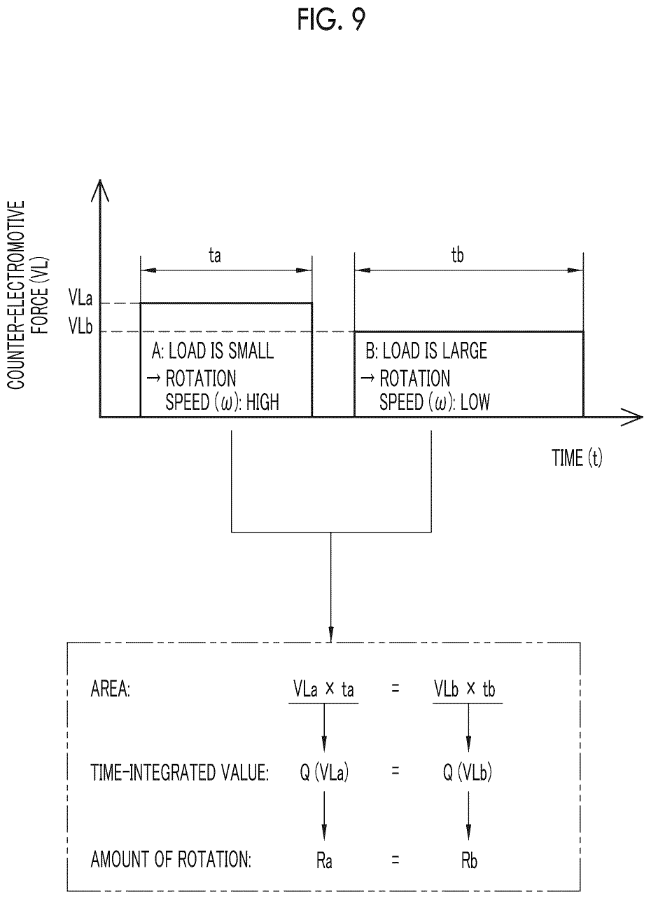

Such effects will be described more specifically with reference to FIG. 9. As shown in FIG. 9, “A” which is a state where a load is relatively small and “B” which is a state where a load is relatively large are compared. In this case, in the state “A” where the load is small, the rotation speed ω of the DC motor 43 is high, and a counter-electromotive force VLa is also large. On the contrary, in the state “B” where the load is large, the rotation speed ω of the DC motor 43 is low, and a counter-electromotive force VLb is also small.

In FIG. 9, since the area of each of the rectangles “A” and “B” is a value obtained by multiplying the counter-electromotive force VL proportional to the rotation speed ω by time, the area corresponds to the amount of rotation of the DC motor 43 and the amount of movement of the variable magnification lens 51B corresponding to the amount of rotation. The area of the rectangle of “A” is the counter-electromotive force VLa×time ta and corresponds to the time-integrated value Q (VLa) of the counter-electromotive force VLa. This time-integrated value Q (VLa) is the amount of rotation Ra of the DC motor 43 and corresponds to the amount of movement of the variable magnification lens 51B. The area of the rectangle of “B” is the counter-electromotive force VLb×time tb and corresponds to the time-integrated value Q (VLb) of the counter-electromotive force VLb. This time-integrated value Q (VLb) is the amount of rotation Rb of the DC motor 43 and corresponds to the amount of movement of the variable magnification lens 51B. The drive time ta of “A” is shorter than the drive time tb of “B”.

Even in a case where the same driving pulse is given to the DC motor 43 by the same number of times in the same period, the rotation speed ω of the DC motor 43 fluctuates as in “A” and “B” due to load fluctuations. Even in this case, since the processor 61 controls the time-integrated value Q (VL) of the counter-electromotive force VL having a correlation with the rotation speed w, the amount of rotation of the DC motor 43 can be accurately learned even in a case where load fluctuations occur. Accordingly, with the lens position control device of the embodiment, it is possible to accurately learn the amount of movement of the variable magnification lens 51B, and the lens position can be controlled with high accuracy. In addition, the processor 61 acquires the average voltage Vav obtained by averaging the drive voltage VD and the counter-electromotive force VL and acquires the time-integrated value Q (VL) of the counter-electromotive force VL based on the average voltage Vav and a known driving pulse corresponding to the drive voltage VD. Since the average voltage Vav can be relatively easily acquired by using the ADC 64 or the like, in some cases, there is an advantage that the configuration is simple by acquiring the time-integrated value Q (VL) from the average voltage Vav.

In addition, the lens position control device of the embodiment comprises an LPF 63 that outputs an output signal of the average voltage Vav obtained by smoothing an input signal corresponding to each of waveforms of the drive voltage VD and the counter-electromotive force VL generated between the terminals of the DC motor 43 to the processor. With the LPF 63, since noise of a signal waveform can also be removed, a signal(S)/noise (N) ratio can be improved, it is possible to control the lens position with higher accuracy.

In addition, in the lens position control device of the embodiment, the processor 61 performs step zoom control of stepwise changing the focal length of the zooming optical system 51 by stepwise changing the position of the variable magnification lens 51B to the plurality of target positions set in advance. In a case of performing such step zoom control, control of the lens position with high accuracy is required. Therefore, the present disclosed technology is particularly effective.

In addition, in the embodiment, since the processor 61 controls the position of the variable magnification lens 51B using table data, such as the lens target position information shown in FIG. 7, the processing can be simplified compared to a case of not using the lens target position information. It is evident that the correspondence relationship between the target position, such as each of the step positions SP1 to SP5, and the target value TQ (VL) of the time-integrated value, which is recorded as the lens target position information, may not be in the form of table data and may be stored in the memory 65 in the form of a function.

In addition, in the embodiment, the drive force transmission mechanism including the torque wire 42 and the reducer 44 is used. By using the torque wire 42 in such a drive force transmission mechanism, it is possible to control the lens position of the zooming optical system 51 at the distal end portion 23 of the small-diameter insertion part 17. In addition, the torque wire 42 is less twisted by the reducer 44. For this reason, such a drive force transmission mechanism is effective for the endoscope 10.

Further, as shown in FIG. 2, in the embodiment, the variable magnification lens 51B moves from the wide end to the telephoto end in a range in which the amount of rotation of the output shaft 45 is within one rotation. Accordingly, the torque wire 42 can be made even less twisted.

### Modification Example of First Embodiment

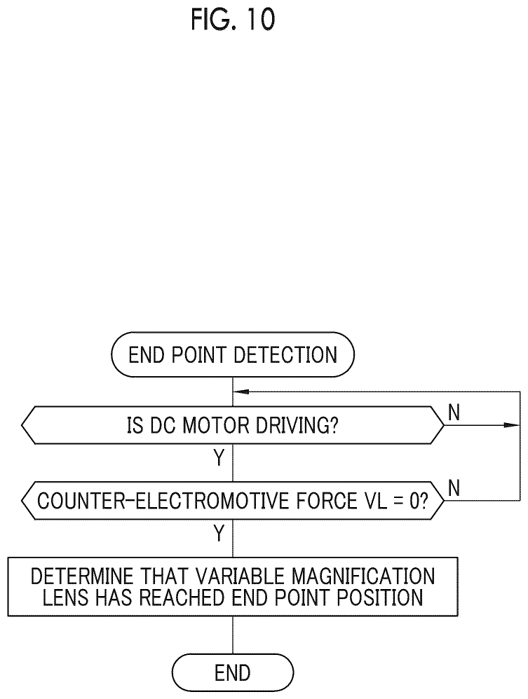

As shown in FIG. 10, the processor 61 may monitor the value of the counter-electromotive force VL while driving the DC motor 43 and detect that the variable magnification lens 51B has reached the wide end or the telephoto end in a case where the value of the counter-electromotive force VL is a value of a rotation stop state of the DC motor 43. A configuration that is a premise for a case of performing such end point detection is a configuration where the output shaft 45 stops rotating by touching the touching portion 56 at each of the wide side end point position PW and the telephoto side end point position PT as shown in FIG. 2.

In a case where the output shaft 45 touches the touching portion 56 during the driving of the DC motor 43, the rotation of the DC motor 43 also stops. In a case where the rotation of the DC motor 43 stops, it is evident that the counter-electromotive force VL generated at the DC motor 43 is also “0”. This value is an example of a value of the rotation stop state. In a case where the counter-electromotive force VL is “0”, the processor 61 can determine that the variable magnification lens 51B has reached any one of the wide side end point position PW or the telephoto side end point position PT by monitoring the counter-electromotive force VL. With this determination, it is possible to detect the end point of the output shaft 45. Each end point position of the output shaft 45 corresponds to the wide end or the telephoto end of the variable magnification lens 51B. For this reason, the processor 61 can detect that the variable magnification lens 51B has reached the wide end or the telephoto end through the end point detection of the output shaft 45.

### Second Embodiment

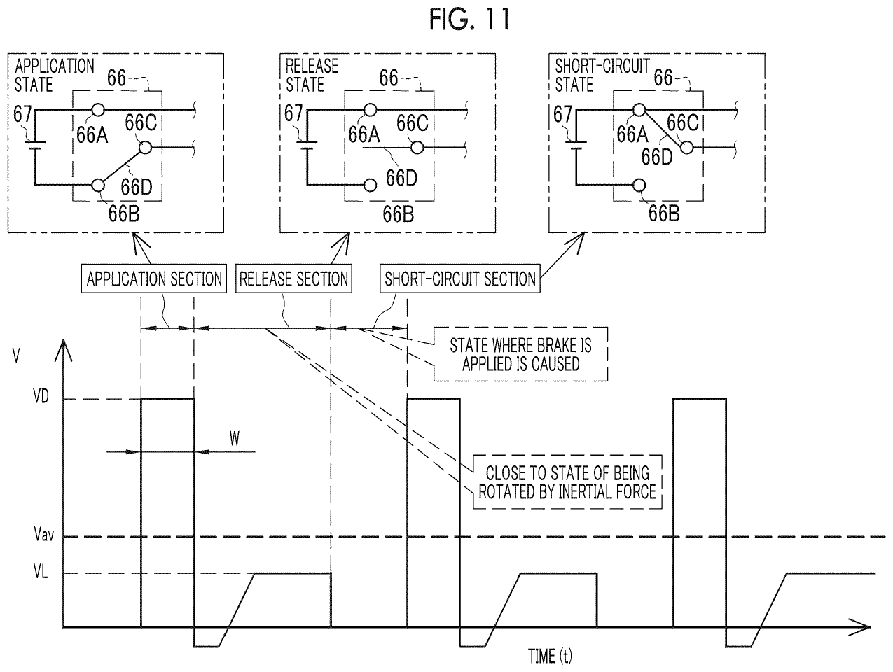

As in a second embodiment shown in FIG. 11, the drive circuit 62 may drive the DC motor 43 by repeating three states including the short-circuit state for short-circuiting between the terminals of the DC motor 43, in addition to the application state and the release state. The short-circuit state is a state where one end of the switch 66D is connected to the first contact 66A and the terminals of the DC motor 43 are short-circuited. As shown in the time chart of FIG. 11, in a case where a reference voltage is “0”, the terminal-to-terminal voltage V is “O” in the short-circuit state.

In the release section corresponding to the release state, the counter-electromotive force VL is generated, but the DC motor 43 is nearly in a state of being rotated by the inertial force generated in the application section. For this reason, in the release section, fluctuations in the rotation speed ω of the DC motor 43 caused by load fluctuations are likely to occur. The fluctuations in the rotation speed ω affect the movement time of the variable magnification lens 51B.

On the other hand, in a short-circuit section corresponding to the short-circuit state, the DC motor 43 is in a state where a brake is applied. For this reason, by adding the short-circuit state, a load fluctuation resistance (that is, a resistance to the load fluctuations) can be improved.

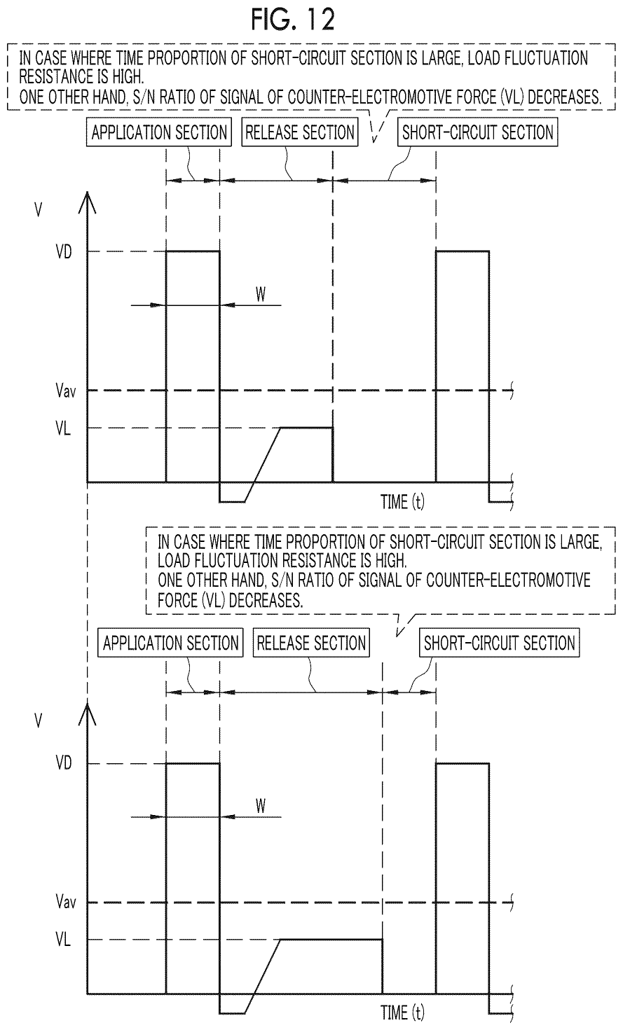

As shown in the time chart in the upper part of FIG. 12, the load fluctuation resistance is improved in a case where the time proportion of the short-circuit section is large. On the other hand, since the time proportion of the release section where the counter-electromotive force VL is generated is large, the S/N ratio of a signal of the counter-electromotive force VL is improved.

As described above, even in a case where the short-circuit section is mixed in the period of a driving pulse, there are advantages and disadvantages depending on the sizes of the time proportions of the short-circuit section and the open section. Therefore, it is preferable to determine the time proportions in consideration thereof. For example, it is preferable that the time proportion of the short-circuit section is set in a range of 30% to 70% with respect to the total time of the release section and the short-circuit section. This is also because it is considered that the S/N ratio between the load fluctuation resistance and the counter-electromotive force VL can be secured in a good balance.

### Third Embodiment

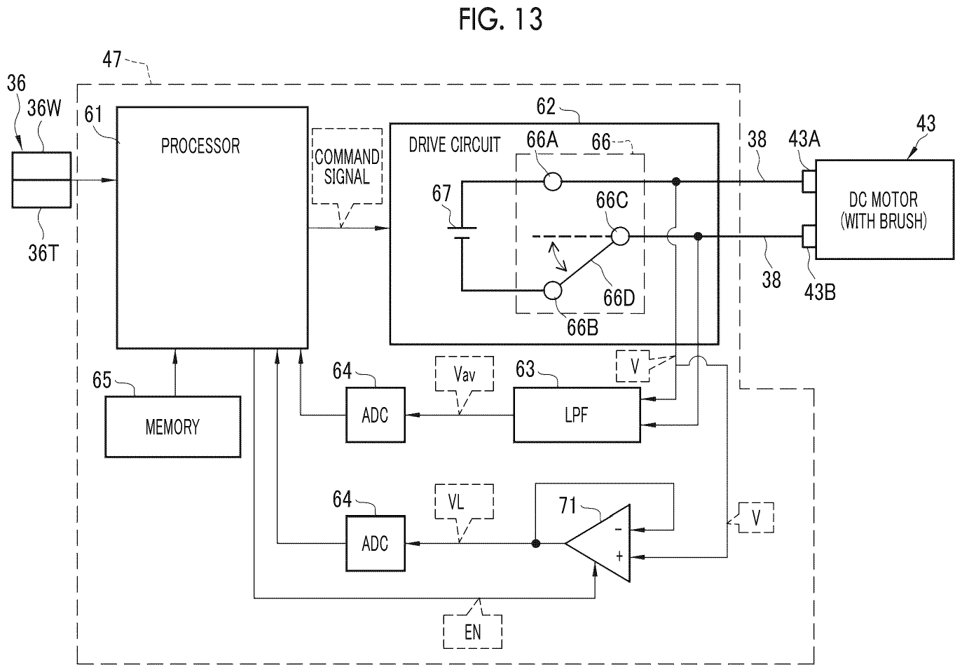

A third embodiment shown in FIG. 13 has a form in which the counter-electromotive force VL is monitored and the counter-electromotive force VL is used in control. In the example shown in FIG. 10, the counter-electromotive force VL is used in end point detection. In the third embodiment, the counter-electromotive force VL is used in various types of control, for example, various types of control including speed control and abnormality detection.

In the third embodiment shown in FIG. 13, in order to actively use the counter-electromotive force VL in various types of control, a circuit configuration for detecting the counter-electromotive force VL is provided instead of acquiring the counter-electromotive force VL from the average voltage Vav as in the first embodiment. Specifically, as shown in FIG. 13, an operational amplifier 71 is provided in addition to the LPF 63. The terminal-to-terminal voltage V of the DC motor 43 is input to a non-reversing input terminal of the operational amplifier 71. In addition, an output of the operational amplifier 71 is input to a reversing input terminal. Accordingly, a voltage buffer circuit that reduces an effect caused by a difference in impedance between an input side and an output side of the operational amplifier 71 is configured.

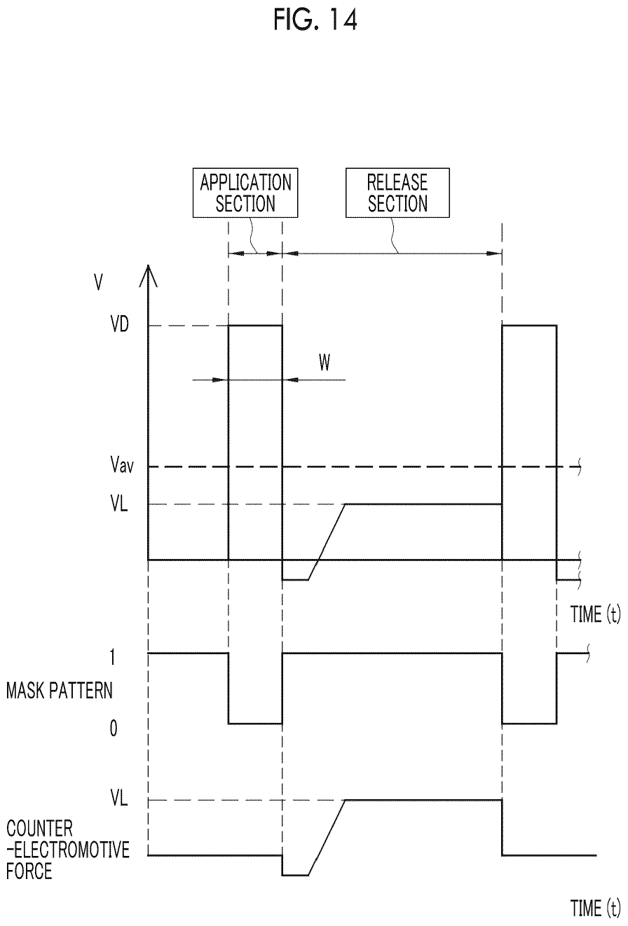

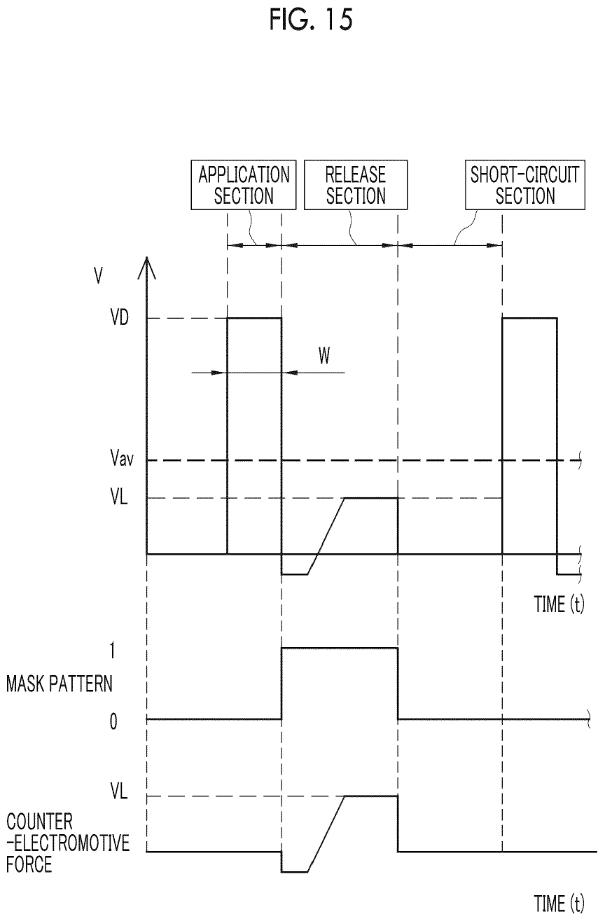

An enable signal (indicated by EN in FIG. 13) is input to the operational amplifier 71 from the processor 61. As shown in FIGS. 14 and 15, the enable signal functions as a mask pattern for extracting the counter-electromotive force VL from the terminal-to-terminal voltage V.

FIG. 14 is an example in which a mask pattern is applied to the drive circuit 62 of the first embodiment that repeats two states including the application state and the release state shown in FIG. 4. FIG. 15 is an example in which a mask pattern is applied to the drive circuit 62 that repeats three states including the application state, the release state, and the short-circuit state shown in FIGS. 11 and 12 of the second embodiment. In either case, a signal of the counter-electromotive force VL is extracted by the mask pattern that is turned on (indicated by “1”) in the release section.

The ADC 64 that converts an analog signal of the counter-electromotive force VL into a digital signal is provided in the subsequent stage of the operational amplifier 71, and the digital signal of the counter-electromotive force VL is input to the processor 61 from the ADC 64. The processor 61 monitors the value of the counter-electromotive force VL.

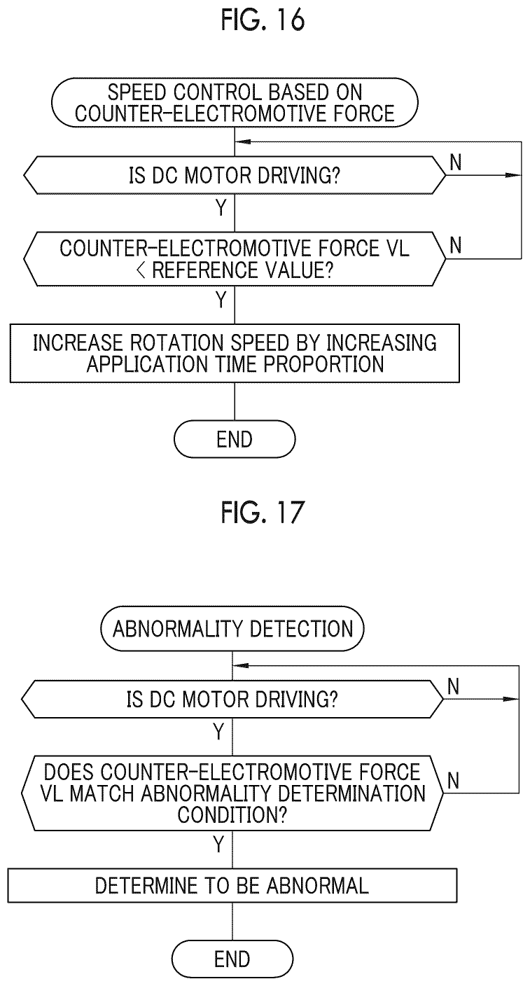

FIG. 16 is an example in which the counter-electromotive force VL is used in speed control of the DC motor 43. As shown in FIG. 16, the processor 61 compares the counter-electromotive force VL to a reference value set in advance while the DC motor 43 is driving. Then, based on the comparison result, the rotation speed ω of the DC motor 43 is controlled by adjusting an application time proportion of a drive voltage through the drive circuit 62. The larger the application time proportion of the drive voltage, the larger the duty ratio, and the smaller the application time proportion, the smaller the duty ratio. For example, in a case where the counter-electromotive force VL falls below the reference value, the processor 61 increases the application time proportion of the drive voltage VD by increasing the pulse width W of the driving pulse in control of the rotation speed. Accordingly, the rotation speed w of the DC motor 43 can be increased.

Since the counter-electromotive force VL represents the actual rotation speed of the DC motor 43, it is possible to perform highly accurate speed control by controlling the rotation speed ω based on the counter-electromotive force VL.

FIG. 17 is an example in which the counter-electromotive force VL is used in abnormality detection. In this case, determination conditions of the abnormality detection are stored in advance in the memory 65. As the abnormality detection, for example, disconnection of the signal line 38 between the DC motor 43 and the drive circuit 62 or the like may be used in addition to an abnormality of the DC motor 43 itself.

As an abnormality of the DC motor 43, there is an abnormality caused by performance deterioration of the DC motor 43. Elements of the performance deterioration of the DC motor 43 include an increase in a sliding load caused by abrasion of gears, bearings, brushes, and commutators and generation of an oxide film, abrasive powder, foreign matters between the brushes and the commutators. These abnormalities can be detected based on the value of the counter-electromotive force VL.

For example, in a case where an increase in a sliding load is caused, the number of rotations of the DC motor 43 decreases in the release section. For this reason, in a case where the counter-electromotive force VL having a value lower than the value of the assumed counter-electromotive force VL is detected, the processor 61 can determine that a load equal to or larger than the assumed load is generated in the DC motor 43. In a case where the rotation of the DC motor 43 is locked and stopped due to overload, the counter-electromotive force VL is not generated, and thus, the terminal-to-terminal voltage V in the release section is “0”.

In addition, in a case where the signal line 38 between the drive circuit 62 and the DC motor 43 is disconnected, the terminal-to-terminal voltage V is a constant value corresponding to the drive voltage VD due to a pull-down (or pull-up) resistor without distinction between the application section and the release section. For this reason, the value of the counter-electromotive force VL to be monitored also is a constant value corresponding to the drive voltage VD. Since this value is a value that is greatly deviated from the value of the counter-electromotive force VL detected at normal times, it is possible to easily detect disconnection. In addition, in the DC motor 43 with a brush, an instantaneous insulation state is caused in some cases due to foreign matters generated between the brush and the commutator. Since the value of the counter-electromotive force VL to be monitored is a constant value corresponding to the drive voltage VD even in such an insulation state, it is possible to detect the disconnection in the same manner.

As shown in FIG. 1, in the case of the endoscope 10, since the signal line 38 is provided at the universal cord 19 or the like, there is a risk of disconnection due to bending or the like. For this reason, such a technique is particularly effective in the case of the endoscope 10.

In a case where the counter-electromotive force VL is used in abnormality detection as described above, a threshold value of the counter-electromotive force VL for determining an abnormality is stored in advance in the memory 65 as an abnormality determination condition. As shown in FIG. 17, the processor 61 determines whether or not the monitored counter-electromotive force VL matches the abnormality determination condition while the DC motor 43 is driving and determines that there is an abnormality in a case where the counter-electromotive force VL matches the abnormality determination condition. In a case where the processor 61 determines that there is an abnormality, the processor 61 issues a warning of the abnormality. The warning may be performed through various indicators in addition to being displayed on the monitor 13. It is evident that voice can also be used.

In addition, although an example in which the counter-electromotive force VL is directly acquired by the operational amplifier 71 has been described in the third embodiment, the counter-electromotive force VL may be indirectly acquired from the average voltage Vav as in the first embodiment.

The various types of control described in the third embodiment can also be performed by comparing the average voltage Vav to a reference value set in advance. This is because the average voltage Vav is a value in which the counter-electromotive force VL is reflected. As described above, a method of using the average voltage Vav as it is can be considered as a so-called method of indirectly using the counter-electromotive force VL. However, in this case, the S/N ratio of a signal value of the counter-electromotive force VL decreases. For this reason, a method of acquiring the counter-electromotive force VL with the operational amplifier 71 or the like as described in the third embodiment or a method of acquiring the counter-electromotive force VL by calculating the counter-electromotive force VL from the average voltage Vav as described in the first embodiment has a high S/N ratio compared to a method of directly using the average voltage Vav. Therefore, it is possible to perform control with higher accuracy.

From the description, the technique described in the following supplementary notes can be learned.

### Supplementary Note 1

A lens position control device that controls a position of a variable magnification lens of a zooming optical system provided at an endoscope, the device comprising:
a DC motor with a brush that generates a drive force for changing the position of the variable magnification lens;
a drive circuit that drives the DC motor with a brush by repeating an application state where a drive voltage is applied between a pair of terminals of the DC motor with a brush and a release state where the terminals are released from each other; and
a processor configured to control the position of the variable magnification lens based on a time-integrated value of a counter-electromotive force generated at the DC motor with a brush in the release state.

### Supplementary Note 2

The lens position control device according to supplementary note 1,
in which the processor is configured to:
acquire an average voltage obtained by averaging the drive voltage and the counter-electromotive force and acquire the time-integrated value of the counter-electromotive force based on the average voltage and a known driving pulse corresponding to the drive voltage.

### Supplementary Note 3

The lens position control device according to supplementary note 2, further comprising:
a low-pass filter that outputs an output signal of the average voltage obtained by smoothing an input signal corresponding to each of waveforms of the drive voltage generated between the terminals and the counter-electromotive force to the processor.

### Supplementary Note 4

The lens position control device according to any one of supplementary notes 1 to 3,
in which the processor is configured to:
perform step zoom control of stepwise changing a focal length of the zooming optical system by stepwise changing the position of the variable magnification lens to a plurality of target positions set in advance.

### Supplementary Note 5

The lens position control device according to supplementary note 4,
in which a correspondence relationship between the plurality of target positions of the variable magnification lens and the time-integrated value of the counter-electromotive force required in order to move the variable magnification lens to each of the target positions is stored in advance in a memory, and
the processor is configured to:
control the position of the variable magnification lens based on the correspondence relationship acquired from the memory.

### Supplementary Note 6

The lens position control device according to any one of supplementary notes 1 to 5, further comprising:
a drive force transmission mechanism including a torque wire that transmits the drive force of the DC motor with a brush to the variable magnification lens and a reducer that decelerates rotation of the DC motor with a brush to transmit the rotation to the torque wire and that has an output shaft which is connected to the torque wire and which rotates the torque wire about an axis.

### Supplementary Note 7

The lens position control device according to supplementary note 6,
in which the variable magnification lens moves from a wide end to a telephoto end in a range in which an amount of rotation of the output shaft is within one rotation.

### Supplementary Note 8

The lens position control device according to supplementary note 7, further comprising:
in a case where a rotational position of the output shaft in a case where the variable magnification lens is at one of the wide end or the telephoto end is defined as a first end point position, and the rotational position of the output shaft in a case where the variable magnification lens is at the other is defined a second end point position, a touching portion that regulates rotation of the output shaft by touching the output shaft at each of the first end point position and the second end point position,
in which the processor is configured to:
monitor the counter-electromotive force while the DC motor with a brush is driving and detect that the variable magnification lens has reached the wide end or the telephoto end in a case where the counter-electromotive force reaches a value of a rotation stop state of the DC motor with a brush.

### Supplementary Note 9

The lens position control device according to any one of supplementary notes 1 to 8,
in which the drive circuit drives the DC motor with a brush by repeating three states including a short-circuit state where the terminals are short-circuited, in addition to the application state and the release state.

### Supplementary Note 10

The lens position control device according to any one of supplementary notes 1 to 9,
in which the processor is configured to:
monitor the counter-electromotive force while the DC motor with a brush is driving and use the counter-electromotive force in control.

### Supplementary Note 11

The lens position control device according to supplementary note 10,
in which the processor is configured to:
control a rotation speed of the DC motor with a brush by adjusting an application time proportion of the drive voltage through the drive circuit based on a result of comparing the counter-electromotive force to a reference value set in advance.

### Supplementary Note 12

The lens position control device according to supplementary note 11,
in which the processor is configured to:
detect an abnormality including at least one of an abnormality of the DC motor with a brush or disconnection of a wiring line between the drive circuit and the DC motor with a brush based on the counter-electromotive force.

### Supplementary Note 13

An endoscope apparatus comprising:
an endoscope that is provided with a zooming optical system; and
a lens position control device that controls a position of a variable magnification lens of the zooming optical system,
in which the lens position control device includes
   a DC motor with a brush that generates a drive force for changing a position of the variable magnification lens,
   a drive circuit that drives the DC motor with a brush by repeating an application state where a drive voltage is applied between a pair of terminals of the DC motor with a brush and a release state where the terminals are released from each other, and
   a processor configured to control the position of the variable magnification lens based on a time-integrated value of a counter-electromotive force generated at the DC motor with a brush in the release state.

In each of the embodiments, for example, as a hardware structure of a processing unit that executes various types of processing, such as the processor 61, various types of processors shown below can be used. Examples of the various types of processors include a central processing unit (CPU) that is a general-purpose processor functioning as the various types of processing units by executing software, a programmable logic device (PLD) such as a field programmable gate array (FPGA) that is a processor having a circuit configuration changeable after manufacture, and/or a dedicated electric circuit such as an application specific integrated circuit (ASIC) that is a processor having a circuit configuration designed for exclusive use in order to execute specific processing.

One processing unit may be composed of one of the various types of processors or may be composed of a combination of two or more processors of the same type or different types (for example, a combination of a plurality of FPGAs and/or a combination of a CPU and an FPGA). As described above, as a hardware structure, various types of processing units are configured by using one or more of the various processors.

Further, as the hardware structure of the various types of processors, more specifically, an electric circuit (circuitry) in which circuit elements such as semiconductor elements are combined can be used.

In addition, in addition to the lens position control device and the endoscope apparatus, the present disclosed technology also includes, in a case where these apparatuses are realized by an operation program, the operation program. Further, in addition to the operation program, the present disclosed technology also includes a non-transitory computer readable storage medium (a USB memory, a digital versatile disc (DVD)-read only memory (ROM), or the like) storing the operation program.

The content of the description made hereinbefore and the content of the drawings are detailed description of portions according to the present disclosed technology and are merely examples of the present disclosed technology. For example, description related to the configurations, functions, actions, and effects is description related to an example of configurations, functions, actions, and effects of the portions according to the present disclosed technology. Accordingly, it is needless to say that unnecessary portions may be deleted, new elements may be added, or replacements may be made with respect to the content of the description made hereinbefore and the content of the drawings within a range that does not deviate from the gist of the present disclosed technology. In addition, in order to avoid confusion and to facilitate understanding of the portions according to the present disclosed technology, description related to common technical knowledge and the like that do not require particular description to enable implementation of the present disclosed technology is omitted from the content of the description made hereinbefore and the content of the drawings.

In the present specification, “A and/or B” is synonymous with “at least one of A or B.” That is, “A and/or B” means that it may be only A, only B, or a combination of A and B. In addition, in the present specification, also in a case where three or more matters are expressed by “and/or” in combination, the same concept as “A and/or B” is applied.

All publications, patent applications, and technical standards described in the present specification are incorporated herein by reference to the same extent as in a case where each publication, each patent application, and each technical standard are specifically and individually indicated to be incorporated by reference.

### EXPLANATION OF REFERENCES

What is claimed is:

1. A lens position control device that controls a position of a variable magnification lens of a zooming optical system provided at an endoscope, the device comprising:
a DC motor with a brush that generates a drive force for changing the position of the variable magnification lens;
a drive circuit that drives the DC motor with a brush by repeating an application state where a drive voltage is applied between a pair of terminals of the DC motor with a brush and a release state where the terminals are released from each other; and
a processor configured to control the position of the variable magnification lens based on a time-integrated value of a counter-electromotive force generated at the DC motor with a brush in the release state.

2. The lens position control device according to claim 1,
wherein the processor is configured to:
acquire an average voltage obtained by averaging the drive voltage and the counter-electromotive force and acquire the time-integrated value of the counter-electromotive force based on the average voltage and a known driving pulse corresponding to the drive voltage.

3. The lens position control device according to claim 2, further comprising:
a low-pass filter that outputs an output signal of the average voltage obtained by smoothing an input signal corresponding to each of waveforms of the drive voltage generated between the terminals and the counter-electromotive force to the processor.

4. The lens position control device according to claim 1,
wherein the processor is configured to:
perform step zoom control of stepwise changing a focal length of the zooming optical system by stepwise changing the position of the variable magnification lens to a plurality of target positions set in advance.

5. The lens position control device according to claim 4,
wherein a correspondence relationship between the plurality of target positions of the variable magnification lens and the time-integrated value of the counter-electromotive force required in order to move the variable magnification lens to each of the target positions is stored in advance in a memory, and
the processor is configured to:
control the position of the variable magnification lens based on the correspondence relationship acquired from the memory.

6. The lens position control device according to claim 1, further comprising:
a drive force transmission mechanism including a torque wire that transmits the drive force of the DC motor with a brush to the variable magnification lens and a reducer that decelerates rotation of the DC motor with a brush to transmit the rotation to the torque wire and that has an output shaft which is connected to the torque wire and which rotates the torque wire about an axis.

7. The lens position control device according to claim 6,
wherein the variable magnification lens moves from a wide end to a telephoto end in a range in which an amount of rotation of the output shaft is within one rotation.

8. The lens position control device according to claim 7, further comprising:
in a case where a rotational position of the output shaft in a case where the variable magnification lens is at one of the wide end or the telephoto end is defined as a first end point position, and the rotational position of the output shaft in a case where the variable magnification lens is at the other is defined a second end point position, a touching portion that regulates rotation of the output shaft by touching the output shaft at each of the first end point position and the second end point position,
wherein the processor is configured to:
monitor the counter-electromotive force while the DC motor with a brush is driving and detect that the variable magnification lens has reached the wide end or the telephoto end in a case where the counter-electromotive force reaches a value of a rotation stop state of the DC motor with a brush.

9. The lens position control device according to claim 1,
wherein the drive circuit drives the DC motor with a brush by repeating three states including a short-circuit state where the terminals are short-circuited, in addition to the application state and the release state.

10. The lens position control device according to claim 1,
wherein the processor is configured to:
monitor the counter-electromotive force while the DC motor with a brush is driving and use the counter-electromotive force in control.

11. The lens position control device according to claim 10,
wherein the processor is configured to:
control a rotation speed of the DC motor with a brush by adjusting an application time proportion of the drive voltage through the drive circuit based on a result of comparing the counter-electromotive force to a reference value set in advance.

12. The lens position control device according to claim 10,
wherein the processor is configured to:
detect an abnormality including at least one of an abnormality of the DC motor with a brush or disconnection of a wiring line between the drive circuit and the DC motor with a brush based on the counter-electromotive force.

13. An endoscope apparatus comprising:
an endoscope that is provided with a zooming optical system; and
a lens position control device that controls a position of a variable magnification lens of the zooming optical system,
wherein the lens position control device includes
a DC motor with a brush that generates a drive force for changing the position of the variable magnification lens,
a drive circuit that drives the DC motor with a brush by repeating an application state where a drive voltage is applied between a pair of terminals of the DC motor with a brush and a release state where the terminals are released from each other, and
a processor configured to control the position of the variable magnification lens based on a time-integrated value of a counter-electromotive force generated at the DC motor with a brush in the release state.
